(19) United States
(12) Patent Application Publication
(10) Pub. No.: US 2025/0064469 A1
(43) Pub. Date: Feb. 27, 2025

(54) ULTRASOUND TRANSDUCER AND ARRAY FOR INTRAVASCULAR THROMBOLYSIS

(71) Applicants: NORTH CAROLINA STATE UNIVERSITY, Raleigh, NC (US); THE UNIVERSITY OF NORTH CAROLINA AT CHAPEL HILL, Chapel Hill, NC (US)

(72) Inventors: Xiaoning Jiang, Cary, NC (US); Jinwook Kim, Raleigh, NC (US); Jianguo Ma, Raleigh, NC (US); Xuming Dai, Chapel Hill, NC (US)

(21) Appl. No.: 18/763,211

(22) Filed: Jul. 3, 2024

Related U.S. Application Data
(63) Continuation of application No. 16/317,983, filed on Jan. 15, 2019, now abandoned.
(60) Provisional application No. 62/362,687, filed on Jul. 15, 2016.

(51) Int. Cl. A61B 17/22; A61B 18/00; A61B 18/22; A61B 18/26

(52) U.S. Cl. CPC A61B 17/2202; A61B 18/26; A61B 2017/22028; A61B 2017/22084; A61B 2017/22089; A61B 2018/00994; A61B 2018/2266; A61B 2018/266

(57) ABSTRACT

A catheter-implemented transducer device for intravascular thrombolysis, is described herein. Such a transducer device includes a catheter defining a longitudinal axis and having opposed proximal and distal ends. At least one ultrasonic transducer arrangement is disposed about the distal end. The ultrasonic transducer arrangement is oriented with acoustic waves propagating parallel or perpendicular to the longitudinal axis. Optionally, the ultrasonic transducer arrangement is configured as a multi-layer stacked structure of ultrasonic transducer elements. Optionally, the ultrasonic transducer arrangement is a laser ultrasonic transducer arrangement. Optionally, the ultrasonic transducer arrangement is configured to operate in a lateral mode.

### CROSS-REFERENCE TO RELATED APPLICATIONS

This application claims the benefit of U.S. provisional patent application No. 62/362,687, filed on Jul. 15, 2016, and entitled “Hybrid Ultrasound Transducer and Array for Intravascular Thrombolysis,” the disclosure of which is expressly incorporated herein by reference in its entirety.

### STATEMENT REGARDING FEDERALLY FUNDED RESEARCH

This invention was made with government support under grant number EB015508 awarded by the National Institutes of Health. The government has certain rights to this invention.

### BACKGROUND

### Field of the Disclosure

The present disclosure is directed to a catheter-implemented transducer device for intravascular thrombolysis.

### Description of Related Art

Deep vein thrombosis or deep venous thrombosis (DVT) is the formation of blood clots within the deep leg veins. The most serious complication of DVT is pulmonary embolism (PE) which is a blockage of a pulmonary artery by a blood clot that detaches from vein walls and travels through the heart to the lungs. Pulmonary embolism (PE) is fatal in more than 100,000 cases annually in the U.S. alone, presents as sudden death in 20-25% of cases, and causes considerable morbidity and health care costs among survivors. Therefore, an effective acute treatment for PE is critically important.

Current PE treatment techniques, such as pharmacological dissolution or fibrinolysis, mechanical fragmentation, and pharmacomechanical thrombolysis, may be hindered by low thrombolysis efficiency, bleeding complications, a relatively high failure rate, vein injury-associated severe regional dysfunction, recurrence, and the risk of distal embolism due to the relatively large size of clot debris. Recent technologies, such as catheter-based side-looking intravascular ultrasound thrombolysis (e.g., EKOS) have somewhat improved performance, but still may suffer from relatively long treatment times (i.e., >10 hours) and concerns about tissue damage from overexposure to acoustic energy. Furthermore, relatively long fluoroscopy times for catheter guidance present some risk to patient and caregiver.

The recombinant tissue-type plasminogen activator (t-PA) has been used for fibrinolysis, but the limitations thereof may include frequent bleeding complications, prolonged infusion time required for the thrombolysis procedure (average 48-53 hours), and high failure rate (about 20%) of fibrinolysis despite the early (within <6 hours) treatment. The mechanical retrieval has been accomplished by using various types of thrombectomy catheters, such as a rotablator, a corkscrew-shaped tip (MERCI), aspiration, rotational, oscillating (Trellis), and rheolytic (Angiojet) thrombectomy. Pharmacomechanical thrombolysis (PMT) has been implemented to use the thrombolytic agent as well as combination of thrombus fragmentation by mechanical devices. Commonly used PMT catheters for relatively large thrombus burden are Angiojet and Trellis. Although these techniques are used to reduce the treatment time with a relatively high success rate, several limitations have been noted, including associated vein injury which leads to severe regional dysfunction, and occurrence of distal embolism due to relatively large size of clot debris.

Ultrasound-based approaches have been developed to overcome these limitations and promote efficiency of thrombolysis, without increasing the risk of systemic bleeding complications. The ‘sonothrombolysis’ approach has exhibited a high benefit-to-risk ratio due to its ability to provide a controlled region of clot dissolution and to resolve clots quickly with limited mechanical contact with either the thrombus or the surrounding vein wall. There are two main mechanisms of the ultrasound-induced techniques: 1) microstreaming, involving jets arising from cavitation adjacent to the clot surface which mechanically cleaves clot fragments; and 2) enhanced penetration of a chemical thrombolytic agent due to the microstreaming.

Ultrasound-delivery methods for thrombolysis (ultrasound-induced thrombolysis) are generally categorized into three techniques: 1) transcutaneous-delivered external ultrasound (TDEU), 2) catheter-delivered external transducer ultrasound (CETU), and 3) catheter-delivered transducer-tipped ultrasound (CTTU) (see, e.g., FIGS. 1A-1C). The TDEU technique is usually accompanied with high-intensity-focused ultrasound (HIFU) for dissolving the clots by cavitation-induced microstreaming. Although this approach is relatively fast, t-PA-free, and noninvasive, there is, for example, potential for damaging the vessel and surround tissue by ultrasound-induced heating due to the large focal spot (i.e., >5 mm) of relatively low-frequency (i.e., <1 MHz) ultrasound energy.

The CETU technique uses low-frequency (i.e., 20-50 kHz) ultrasound waves transmitted through a catheter guide-wire acting as a wave guide. Limitations of this technique include, for example, a narrow bandwidth of usable frequencies, dissipation of ultrasound energy in the wave guide, and increased risk associated with direct contact on the clot. In comparison with other methods, the CTTU technique has exhibited several advantages including, for example, efficient delivery of acoustic energy, flexible frequency control, and negligible ultrasound-induced heating on surrounding tissue. It has been generally accepted that CTTU only facilitates clot dissolution by utilizing low intensity ultrasound to enhance clot permeability to t-PA, which reflects that thrombolysis efficiency of CTTU relies on some amount of t-PA, while the administered t-PA dose must be limited due to potential bleeding complications and strict contraindication criteria.

Currently, a commonly used CTTU technique is the EKOS system (EKOSONIC Endovascular System from EKOS Corporation of Bothell, Washington), which does not fracture or break the thrombus, but uses ultrasound to help loosen the fibrin strands within the clot, allowing deeper penetration of lytic agent and reducing the risk of distal embolism. Although this treatment is characterized by reduced dose of t-PA and treatment time (usually 24-48 hours), in comparison with a conventional catheter-directed thrombolysis (CDT) which usually takes three to five days, it may be desirable to further reduce the t-PA dose and extensive treatment time in order to reduce the risk of hemorrhage and to reduce costs. For higher lytic rate with decreased dose of t-PA, the current limitation of a CTTU technique is the lack of miniaturized (i.e., capable of fitting in a 7-French or smaller catheter), low-frequency ultrasound transducers to generate microstreaming arising from cavitation. Therefore, there exists a need for catheter-based therapy for PE or DVT, for a device which is compact in size and provides sufficient acoustic output for cavitation-induced microstreaming, with a compact focal spot and precise spatiotemporal delivery of a minimal dose of a lytic agent.

Cavitation enhancement involves enhancing the mechanical effect of cavitation-induced microstreaming, through the application of microbubbles. The presence of microbubbles at the clot surface, typically in the form of ultrasound contrast agents, causes a substantially improved lytic rate than without microbubbles. In vivo and in vitro studies with microbubbles for TDEU application have shown more than 100% improved lytic rate than the case without microbubbles. However, it may be desirable to improve (reduce) variation of microbubbles for lytic enhancement under reduced acoustic pressure. Perfluorocarbon nanodroplets are compositionally similar to bubbles, except for involving a perfluorocarbon core in a liquid state. These droplets can be produced at a fraction of the size of microbubbles (i.e., 100-200 nm), and demonstrate improved stability and circulation time. Upon exposure to a sufficient acoustic threshold, these ‘phase change agents’ vaporize, converting to microbubbles. Intravascular administration of perfluorocarbon droplets has been demonstrated to reduce the sonication power required to achieve recanalization to 24±5% of the necessary power without droplets. The benefit of these nanodroplets over microbubbles is twofold: 1) nanodroplets can penetrate into the clot matrix more efficiently than microbubbles, and 2) increased stability of nanodroplets allows them to be delivered via a catheter. In contrast, microbubbles may be challenging to deliver via a catheter due to their pressure sensitivity, and thus microbubbles are typically administered systemically.

A nanodroplet formulation, substantially similar in composition to lipid-encapsulated microbubbles, has been utilized as a contrast agent. This procedure starts with a microbubble preparation, and compresses the microbubbles into droplets. The droplets stay in this form, until exposed to a sufficient acoustic threshold, due to surface tension and bulk nucleation properties of the liquid core. One benefit of this formulation compared to other phase change agents, such as those made with perfluoropentane, is that a low-boiling point gas core, such as perfluoropropane or decafluorobutane, is utilized, and thus can be readily converted to microbubbles at low mechanical indices. Sub-micron agents of perfluoropentane or higher boiling point perfluorocarbons, on the other hand, require substantially more acoustic power, thereby increasing the potential for bioeffects.

Shock wave enhanced lysis is another way to increase the lytic rate of the TDEU technique, namely by using a pulsed laser for laser-enhanced acoustic cavitation. In this regard, the combined excitation of the target clot by HIFU and a 730 nm laser with higher than 27 mJ/cm² input, may result in about 50% higher lytic efficiency. However, the use of laser energy of 27 mJ/cm² for direct exposure of the clot is over the safety limit (26.4 mJ/cm² for 730 nm laser) recommended by the American National Standards Institute (ANSI) for concerns regarding light energy-induced heating or chemical breakdown.

In light of the state of the art, there exists a need for improved technologies for providing safe and effective thrombus treatment.

### SUMMARY OF THE DISCLOSURE

The above and other needs are met by aspects of the present disclosure which, in one aspect, provides a catheter-implemented transducer device for intravascular thrombolysis. Such a transducer device comprises a catheter defining a longitudinal axis and having opposed proximal and distal ends. A first ultrasonic transducer arrangement (piezoelectric) is disposed about the distal end and oriented perpendicularly to the longitudinal axis. A second ultrasonic transducer arrangement (piezoelectric) is disposed about the distal end of the catheter and oriented parallel to the longitudinal axis. A third ultrasonic transducer arrangement (laser) is disposed about the distal end of the catheter and oriented perpendicularly to the longitudinal axis, and/or a supply conduit is arranged along the catheter and is configured to supply microbubbles, droplets, or t-PA outwardly of the first ultrasonic transducer arrangement from the distal end of the catheter. An associated method is also provided.

Alternatively or additionally, the first ultrasonic transducer arrangement includes an array of ultrasonic transducer elements. The array has a lateral dimension and defining an aperture less than a lateral dimension of the catheter. Optionally, each of the plurality of ultrasonic transducer elements is comprised of a piezoelectric ceramic or a piezoelectric relaxor single crystal.

Alternatively or additionally, the first ultrasonic transducer arrangement is configured as a stacked structure of ultrasonic transducer elements operable in a longitudinal mode to emit forward viewing low-frequency ultrasonic energy and to generate pressure.

Alternatively or additionally, the first ultrasonic transducer arrangement is configured operate in a lateral mode to emit forward viewing low-frequency ultrasonic energy within a frequency range of between less than 1 MHz and about 3 MHz.

Alternatively or additionally, the second ultrasonic transducer arrangement includes a plurality of ultrasonic transducer elements arranged about a circumference of the distal end of the catheter. Each of the plurality of ultrasonic transducer elements is oriented parallel to the longitudinal axis. Optionally, each of the plurality of ultrasonic transducer elements is comprised of a piezoelectric ceramic or a piezoelectric relaxor single crystal.

Alternatively or additionally, the second ultrasonic transducer arrangement is configured to operate in a lateral resonance mode emitting side viewing acoustic waves.

Alternatively or additionally, the first and second ultrasonic transducer arrangements are each configured as a stacked structure of transducer elements operable in a lateral mode to cooperate to generate forward viewing and side viewing waves with pressure capable of inducing cavitation about the distal end of the catheter.

Alternatively or additionally, the third ultrasonic transducer arrangement further includes a laser-generated focused ultrasound (LGFU) lens disposed about the distal end of the catheter and oriented perpendicularly to the longitudinal axis with acoustic waves propagating parallel to the longitudinal axis. Optionally, the LGFU lens is configured as a plano or a concave optical lens a laser ultrasound transduction layer. Optionally, the LGFU lens is arranged to share a focal point with the first ultrasonic transducer arrangement.

Alternatively or additionally, the transducer device further includes a micro-optical fiber or fiber bundle that extends along the longitudinal axis of the catheter and into operable engagement with the LGFU lens. The micro-optical fiber or fiber bundle is configured to direct laser light to and through the LGFU lens. The laser light directed through the LGFU lens interacts with the laser ultrasound transduction layer thereof to photoacoustically convert the laser light to ultrasonic energy, and the converted ultrasonic energy cooperates with ultrasonic energy emitted by an ultrasonic transducer arrangement to induce cavitation about the distal end of the catheter.

Alternatively or additionally, the transducer device further includes a supply conduit arranged along the catheter. The supply conduit is configured to supply at least one of droplets, microbubbles, or a pharmaceutical compound outwardly of the at least one ultrasonic transducer arrangement from the distal end of the catheter.

In another aspect, a catheter-implemented transducer device for intravascular thrombolysis is provided. Such a transducer device includes a catheter defining a longitudinal axis and having opposed proximal and distal ends. At least one ultrasonic transducer arrangement is disposed about the distal end. Additionally, the at least one ultrasonic transducer arrangement is configured as a multi-layer stacked structure of ultrasonic transducer elements.

Alternatively or additionally, the at least one ultrasonic transducer arrangement emits low-frequency ultrasonic energy within a frequency range of between less than 1 MHz and about 3 MHz.

Alternatively or additionally, the at least one ultrasonic transducer arrangement emits ultrasonic waves that propagate parallel or perpendicular to the longitudinal axis.

Alternatively or additionally, the at least one ultrasonic transducer arrangement is configured to operate in a lateral or longitudinal mode.

Alternatively or additionally, the at least one ultrasonic transducer arrangement includes a plurality of ultrasonic transducer elements arranged about a circumference of the distal end of the catheter. Each of the plurality of ultrasonic transducer elements is oriented parallel to the longitudinal axis.

Alternatively or additionally, the transducer device further includes at least two ultrasonic transducer arrangements disposed about the distal end of the catheter. The at least two ultrasonic transducer arrangements operate in a lateral or longitudinal mode to cooperate to generate pressure capable of inducing cavitation about the distal end of the catheter.

Alternatively or additionally, the transducer device further includes an acoustic lens arranged adjacent to and outwardly of the at least one ultrasonic transducer arrangement. The acoustic lens is configured to obtain a focused acoustic field generated by the at least one ultrasonic transducer arrangement.

Alternatively or additionally, the transducer device further includes a laser-generated focused ultrasound (LGFU) lens disposed about the distal end of the catheter and oriented perpendicularly to the longitudinal axis. The LGFU lens is arranged to share a focal point with the at least one ultrasonic transducer arrangement.

Alternatively or additionally, the transducer device further includes a supply conduit arranged along the catheter. The supply conduit is configured to supply at least one of droplets, microbubbles, or a pharmaceutical compound outwardly of the at least one ultrasonic transducer arrangement from the distal end of the catheter.

In yet another aspect, a catheter-implemented transducer device for intravascular thrombolysis is provided. Such a transducer device includes a catheter defining a longitudinal axis and having opposed proximal and distal ends. At least one laser ultrasonic transducer arrangement is disposed about the distal end.

Alternatively or additionally, the at least one laser ultrasonic transducer arrangement includes a laser-generated focused ultrasound (LGFU) lens disposed about the distal end and oriented perpendicularly to the longitudinal axis with acoustic waves propagating parallel to the longitudinal axis.

Alternatively or additionally, the LGFU lens is arranged to share a focal point with the at least one laser ultrasonic transducer arrangement.

Alternatively or additionally, the LGFU lens is configured as a plano or a concave optical lens a laser ultrasound transduction layer.

Alternatively or additionally, the transducer device further includes a micro-optical fiber or fiber bundle that extends along the longitudinal axis of the catheter and into operable engagement with the LGFU lens. The micro-optical fiber or fiber bundle is configured to direct laser light to and through the LGFU lens. The laser light directed through the LGFU lens interacts with the laser ultrasound transduction layer thereof to photoacoustically convert the laser light to ultrasonic energy, and the converted ultrasonic energy cooperates with ultrasonic energy emitted by an ultrasonic transducer arrangement to induce cavitation about the distal end of the catheter.

Alternatively or additionally, the transducer device further includes a supply conduit arranged along the catheter. The supply conduit is configured to supply at least one of droplets, microbubbles, or a pharmaceutical compound outwardly of the at least one laser ultrasonic transducer arrangement from the distal end of the catheter.

In yet another aspect, a catheter-implemented transducer device for intravascular thrombolysis is provided. Such a transducer device includes a catheter defining a longitudinal axis and having opposed proximal and distal ends. A first ultrasonic transducer arrangement is disposed about the distal end and oriented perpendicularly to the longitudinal axis. A second ultrasonic transducer arrangement is disposed about the distal end of the catheter and oriented parallel to the longitudinal axis. A supply conduit is arranged along the catheter and is configured to supply microbubbles, droplets, or a pharmaceutical compound outwardly of the first ultrasonic transducer arrangement from the distal end of the catheter.

Alternatively or additionally, the first ultrasonic transducer arrangement includes an array of ultrasonic transducer elements. The array has a lateral dimension and defining an aperture less than a lateral dimension of the catheter. Optionally, each of the plurality of ultrasonic transducer elements is comprised of a piezoelectric ceramic or a piezoelectric relaxor single crystal.

Alternatively or additionally, the first ultrasonic transducer arrangement is configured as a stacked structure of ultrasonic transducer elements operable in a longitudinal mode to emit forward viewing low-frequency ultrasonic energy and to generate pressure.

Alternatively or additionally, the first ultrasonic transducer arrangement is configured operate in a lateral mode to emit forward viewing low-frequency ultrasonic energy within a frequency range of between less than 1 MHz and about 3 MHz.

Alternatively or additionally, the second ultrasonic transducer arrangement includes a plurality of ultrasonic transducer elements arranged about a circumference of the distal end of the catheter. Each of the plurality of ultrasonic transducer elements is oriented parallel to the longitudinal axis. Optionally, each of the plurality of ultrasonic transducer elements is comprised of a piezoelectric ceramic or a piezoelectric relaxor single crystal.

Alternatively or additionally, the second ultrasonic transducer arrangement is configured to operate in a lateral resonance mode emitting side viewing acoustic waves.

Alternatively or additionally, the first and second ultrasonic transducer arrangements are each configured as a stacked structure of transducer elements operable in a lateral mode to cooperate to generate forward viewing and side viewing waves with pressure capable of inducing cavitation about the distal end of the catheter.

Alternatively or additionally, the transducer device further includes a laser ultrasonic transducer arrangement disposed about the distal end and oriented perpendicularly to the longitudinal axis. The laser ultrasonic transducer arrangement further includes a laser-generated focused ultrasound (LGFU) lens disposed about the distal end of the catheter and oriented perpendicularly to the longitudinal axis with acoustic waves propagating parallel to the longitudinal axis. Optionally, the LGFU lens is configured as a plano or a concave optical lens a laser ultrasound transduction layer. Optionally, the LGFU lens is arranged to share a focal point with the first ultrasonic transducer arrangement.

Alternatively or additionally, the transducer device further includes a micro-optical fiber or fiber bundle that extends along the longitudinal axis of the catheter and into operable engagement with the LGFU lens. The micro-optical fiber or fiber bundle is configured to direct laser light to and through the LGFU lens. The laser light directed through the LGFU lens interacts with the laser ultrasound transduction layer thereof to photoacoustically convert the laser light to ultrasonic energy, and the converted ultrasonic energy cooperates with ultrasonic energy emitted by an ultrasonic transducer arrangement to induce cavitation about the distal end of the catheter.

In another aspect, a catheter-implemented transducer device for intravascular thrombolysis is provided. Such a transducer device includes a catheter defining a longitudinal axis and having opposed proximal and distal ends. At least one ultrasonic transducer arrangement is disposed about the distal end. Additionally, the at least one ultrasonic transducer arrangement is configured to operate in a lateral mode.

Alternatively or additionally, the at least one ultrasonic transducer arrangement emits low-frequency ultrasonic energy within a frequency range of between less than 1 MHz and about 3 MHz.

Alternatively or additionally, the at least one ultrasonic transducer arrangement emits ultrasonic waves that propagate parallel or perpendicular to the longitudinal axis.

Alternatively or additionally, the at least one ultrasonic transducer arrangement includes a plurality of ultrasonic transducer elements arranged about a circumference of the distal end of the catheter. Each of the plurality of ultrasonic transducer elements is oriented parallel to the longitudinal axis.

Alternatively or additionally, the transducer device further includes at least two ultrasonic transducer arrangements disposed about the distal end of the catheter. The at least two ultrasonic transducer arrangements operate in a lateral or longitudinal mode to cooperate to generate pressure capable of inducing cavitation about the distal end of the catheter.

Alternatively or additionally, the transducer device further includes an acoustic lens arranged adjacent to and outwardly of the at least one ultrasonic transducer arrangement. The acoustic lens is configured to obtain a focused acoustic field generated by the at least one ultrasonic transducer arrangement.

Alternatively or additionally, the transducer device further includes a laser-generated focused ultrasound (LGFU) lens disposed about the distal end of the catheter and oriented perpendicularly to the longitudinal axis. The LGFU lens is arranged to share a focal point with the at least one ultrasonic transducer arrangement.

Alternatively or additionally, the transducer device further includes a supply conduit arranged along the catheter. The supply conduit is configured to supply at least one of droplets, microbubbles, or a pharmaceutical compound outwardly of the at least one ultrasonic transducer arrangement from the distal end of the catheter.

The aspects, functions and advantages discussed herein may be achieved independently in various example implementations/aspects or may be combined in yet other example implementations/aspects, further details of which may be seen with reference to the following description and drawings.

### BRIEF DESCRIPTION OF THE DRAWINGS

Having thus described the disclosure in general terms, reference will now be made to the accompanying drawings, which are not necessarily drawn to scale, and wherein:

FIGS. 1A-1C schematically illustrate various ultrasound-induced thrombolysis techniques, including (a) transcutaneous-delivered external ultrasound (TDEU) in FIG. 1A; (b) catheter-delivered external transducer ultrasound (CETU) in FIG. 1B; and (c) catheter-delivered transducer-tipped ultrasound (CTTU) in FIG. 1C;

FIG. 2 schematically illustrates a catheter-mounted, small aperture, hybrid, IVUS thrombolysis transducer device, according to one aspect of the present disclosure;

FIG. 3 schematically illustrates a front-firing, piezoelectric stacked-type, flat or focused element, according to one aspect of the present disclosure;

FIG. 4 schematically illustrates a front-firing, LGFU transducer element, according to one aspect of the present disclosure;

FIG. 5 schematically illustrates a dual excitation, catheter-delivered, laser ultrasound thrombolysis (DECLUT) system, according to one aspect of the present disclosure, having a side viewing piezoelectric cylindrical array transducer and a piezoelectric forward viewing flat or focused transducer;

FIG. 6 schematically illustrates a dual excitation, catheter-delivered, laser ultrasound thrombolysis (DECLUT) system, according to one aspect of the present disclosure, having a side viewing piezoelectric cylindrical array transducer and a hybrid forward viewing flat or focused transducer;

FIGS. 7 and 8 schematically illustrate a structure of a piezoelectric (e.g., capable of operation in lateral mode or thickness mode or longitudinal mode) element, according to aspects of the present disclosure, with FIG. 7 illustrating a single layer piezoelectric element and FIG. 8 illustrating a multi-layer stacked structure;

FIG. 9 schematically illustrates intravascular sonothrombolysis using a DECLUT catheter, low-frequency (<1 MHz) burst waves and laser-generated shock waves to generate microstreaming caused by cavitation of injected droplets/microbubbles;

FIGS. 10A-10C schematically illustrate a piezoelectric multi-layer transducer having (a) 6 layers of 255 m thick PZT-5A ceramics and 22 m-thick copper shims as intermediate electrode layers in FIG. 10A; (b) transducers on a 16 gauge needle tip in FIG. 10B; and (c) a measured pressure output with the 20 cycle of sinusoidal voltage input of 60, 90, 120 Vpp at 550 kHz in FIG. 10C;

FIGS. 11A and 11B schematically illustrate a test arrangement and result for a piezoelectric multi-layer transducer involving (a) an in vitro test arrangement using a bovine blood clot stored in a PVC test tube filled with saline water in FIG. 11A; and (b) in vitro test results of a 30 minute treatment with microbubble injection for a clot mass reduction of 50% in FIG. 11B;

FIG. 12 schematically illustrates a multi-frequency piezoelectric transducer arrangement combining a 10 MHz imaging transducer with 500 kHz and 1 MHz therapy transducers;

FIG. 13 schematically illustrates self-A-mode imaging by DECLUT transducer arrangement;

FIGS. 14A-14B schematically illustrates an analysis of a lateral-mode transducer including (a) an ANSYS simulation on wave propagation of a 1.2×1.2×0.3 mm³ PZT-5H lateral mode transducer at its resonance frequency in FIG. 14A; and (b) a calculated axial pressure output profile in FIG. 14B;

FIGS. 15A and 15B schematically illustrate an optical fiber LGFU transducer (a) fixed at a coupler in FIG. 15A; and (b) a measured waveform and frequency spectrum of the optical fiber LGFU transducer with 1.5 mJ laser input in FIG. 15B;

FIGS. 16A and 16B schematically illustrate in vitro thrombolysis tests for a dual-excitation of LGFU and piezo-ultrasound arrangement, including (a) an experimental arrangement for a dual-excitation test in FIG. 16A; and (b) mass loss for each treatment case (P, L, and P+L denote treatment of piezo-ultrasound, LGFU, and dual-excitation of piezo-ultrasound and LGFU, respectively) in FIG. 16B;

FIG. 17 schematically illustrates an integration procedure of an optical fiber LGFU transducer and a multi-layer transducer; and

FIG. 18 schematically illustrates an experimental DECLUT system, according to one aspect of the present disclosure.

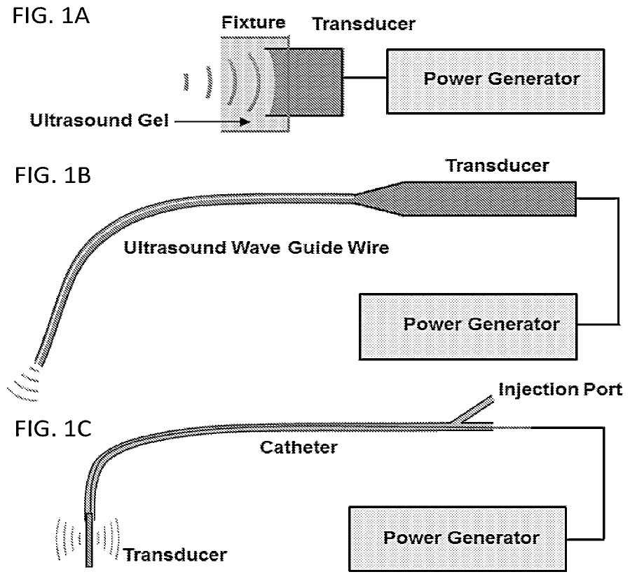

### DETAILED DESCRIPTION OF THE DISCLOSURE

The present disclosure now will be described more fully hereinafter with reference to the accompanying drawings, in which some, but not all aspects of the disclosure are shown. Indeed, the disclosure may be embodied in many different forms and should not be construed as limited to the aspects set forth herein; rather, these aspects are provided so that this disclosure will be thorough and complete, will fully convey the scope of the disclosure to those skilled in the art, and will satisfy applicable legal requirements. Like numbers refer to like elements throughout. As used in this specification and the claims, the singular forms “a,” “an,” and “the” include plural referents unless the context clearly dictates otherwise.

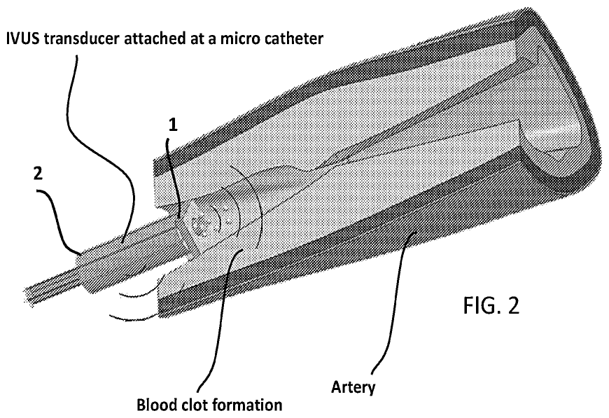

Aspects of the present disclosure are directed to a dual excitation, catheter-delivered, laser ultrasound thrombolysis (DECLUT) system (see, e.g., FIGS. 2 and 9) for improving an intravascular sonothrombolysis procedure. Such a system 100 may, for example, include several devices individually implemented in different approaches to addressing the thrombolysis issue, each of the devices/approaches having demonstrated thrombolysis efficacy in preliminary testing, as well as through other empirical data. Aspects of the present disclosure thus combine certain of these individual devices/approaches in order to, for example, improve lytic rate and reduce treatment time, improve clot lysis performance, and improve safety.

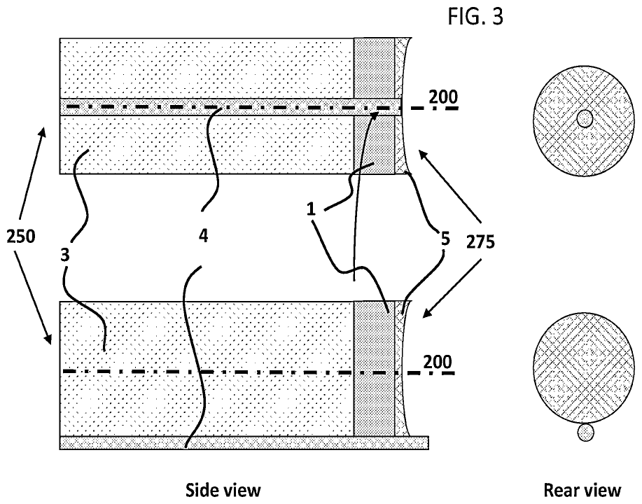

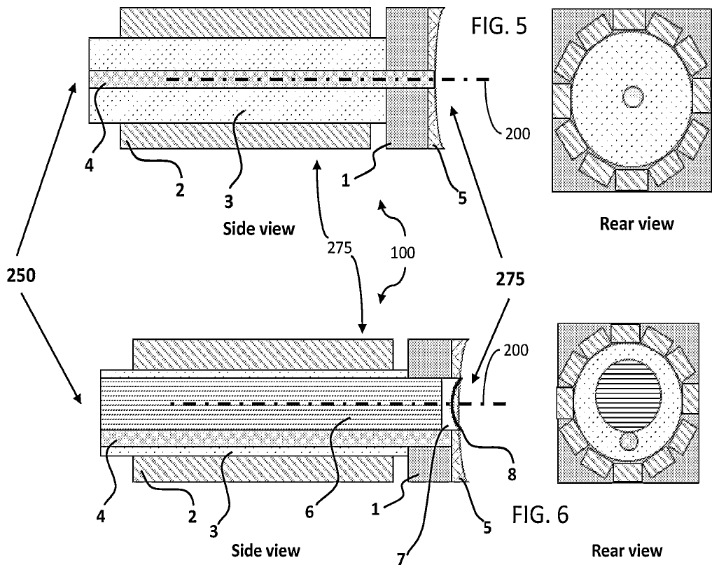

Referring now to FIGS. 3-6, example catheter-implemented transducer devices are described. More particularly, in one aspect, the present disclosure (see, e.g., FIGS. 5 and 6) provides a catheter-implemented transducer device 100 for intravascular thrombolysis. Such a device 100 comprises a catheter 3 defining a longitudinal axis 200 and having opposed proximal and distal ends 250, 275. A first ultrasonic transducer arrangement 1 is disposed about the distal end 275 and is oriented with acoustic waves propagating parallel to the longitudinal axis 200. A second ultrasonic transducer arrangement 2 is disposed about the distal end 275 and is oriented with acoustic waves propagating perpendicular to the longitudinal axis 200. A third ultrasonic transducer arrangement 7 and 8 is disposed about the distal end 275 and is oriented with acoustic waves propagating parallel to the longitudinal axis 200. As described herein, the third ultrasonic transducer arrangement is a laser ultrasonic transducer arrangement, which includes a laser-generated focused ultrasound (LGFU) lens and a coating layer such as a laser ultrasound transduction layer (e.g., light absorption and thermal expansion layers) as described below. As described above, the first, second, and/or third ultrasonic transducer arrangements are arranged about the distal end 275. For example, the first, second, and/or third ultrasonic transducer arrangements can be arranged in proximity to the distal end 275 as shown in the figures. The location of the first, second, and/or third ultrasonic transducer arrangements in the figures are provided only as examples. This disclosure contemplates that the first, second, and/or third ultrasonic transducer arrangements can be arranged near, adjacent to, above, below, to the side, spaced from, etc. relative to the distal end 275. A supply conduit 4 is arranged along the catheter 3 and is configured to supply nanodroplets, microbubbles, t-PA, and/or other blood thinner drug (e.g., pharmaceutical compound) outwardly of the first ultrasonic transducer arrangement 1 from the distal end 275 of the catheter 3. As shown in FIG. 5, the supply conduit 4 is arranged centrally with respect to the catheter 3 (e.g., along the longitudinal axis 200). As shown in FIG. 6, the supply conduit 4 is arranged off-center with respect to the catheter 3 and parallel to the longitudinal axis 200. The arrangements of the supply conduit with respect to the catheter 3 in FIGS. 5 and 6 are provided only as examples. This disclosure contemplates that the supply conduit 4 can be arranged in other locations with respect to the catheter 3.

The first ultrasonic transducer arrangement 1 may comprise an array of ultrasonic transducer elements, the array having a lateral dimension and defining an aperture less than a lateral dimension of the catheter 3. The first ultrasonic transducer arrangement 1 is oriented perpendicular to the longitudinal axis 200 as shown in FIGS. 5 and 6. In some aspects, the first ultrasonic transducer arrangement 1 is configured as a stacked structure of ultrasonic transducer elements (e.g., a multi-layer stacked structure with a plurality of ultrasonic transducer elements) operable in a longitudinal mode to emit low-frequency ultrasonic energy and to generate acoustic pressure. In some aspects, an acoustic lens 5 is arranged adjacent to and outwardly of the transducer 1 to obtain a focused acoustic field generated by the transducer 1 as shown in FIGS. 5 and 6. In some instances, the first ultrasonic transducer arrangement 1 is configured to operate in a lateral mode or in a longitudinal mode to emit low-frequency ultrasonic energy within a frequency range of between about <1 MHz and about 3 MHz.

The second ultrasonic transducer arrangement 2 includes a plurality of ultrasonic transducer elements arranged about a circumference of the distal end 275 of the catheter 3, wherein each of the plurality of ultrasonic transducer elements is oriented parallel to the longitudinal axis 200. Accordingly, the ultrasonic energy emitted by the second ultrasonic transducer arrangement 2 is directed radially outward from the catheter 3. In some aspects, each of the plurality of ultrasonic transducer elements of the first and/or second ultrasonic transducer arrangement 1, 2, is comprised of a PZT ceramic or other piezoelectric materials including, for example, relaxor-PT single crystals and non-lead piezoelectrics. In other aspects, the first and/or second ultrasonic transducer arrangement 1, 2 may be configured to be operable in a lateral resonance mode. In still other aspects, the first and/or second ultrasonic transducer arrangement 1, 2 is/are each configured as a stacked structure of ultrasonic transducer elements operable in a lateral mode or longitudinal mode to cooperate to generate pressure capable of inducing cavitation about the distal end 275 of the catheter 3.

In particular aspects, the device 100 may further include a laser-generated focused ultrasound (LGFU) lens 7 disposed about the distal end 275 of the catheter 3 and oriented perpendicularly to the longitudinal axis 200 as shown in FIG. 6. An LGFU transducer for precise ultrasound therapy may be effective. The LGFU transducer, comprised of a LGFU lens and laser ultrasound transduction layer (e.g., a light absorption layer and a thermal expansion layer) may be capable of generating shock waves with high negative pressure (>10 MPa) at a tight focal spot (<1 mm), which can allow precise control of cavitation, and thus may be effective in intravascular thrombolysis. For example, the LGFU lens 7 is configured as a plano or a concave optical lens coated with a laser ultrasound transduction layer 8. The laser ultrasound transduction layer 8 can include a light absorption layer (e.g., carbon black, carbon nano-fiber film, carbon nanotubes, carbon nano-particles, metal nano-particles) and a thermal expansion layer (e.g., polydimethylsiloxane (PDMS) or other polymers or plastics or other thermoelastic material). In some aspects, the LGFU lens 7 is arranged and configured to share a focal point with the acoustic lens 5 of the first ultrasonic transducer arrangement 1. A micro-optical fiber (or fiber bundle) 6 may also extend along the longitudinal axis 200 of the catheter 3 and into operable engagement with the LGFU lens 7. The micro-optical fiber 6 may be configured to direct laser light to and through the LGFU lens 7, wherein the laser light directed through the LGFU lens 7 is absorbed by the laser ultrasound transduction layer 8 and photoacoustically converted to ultrasonic energy, which cooperates with ultrasonic energy emitted by the first ultrasonic transducer arrangement 1 to induce cavitation about the distal end 275 of the catheter 3.

In another aspect, the present disclosure (see, e.g., FIG. 3) provides a front-firing, piezoelectric stacked-type, flat or focused element for intravascular thrombolysis. Such a device comprises a catheter 3 defining a longitudinal axis 200 and having opposed proximal and distal ends 250, 275. A first ultrasonic transducer arrangement 1 is disposed about the distal end 275 and is oriented with acoustic waves propagating parallel to the longitudinal axis 200. Additionally, an acoustic lens 5 is arranged adjacent to and outwardly of the transducer 1 to obtain a focused acoustic field generated by the transducer 1. Additionally, a supply conduit 4 is arranged along the catheter 3 and is configured to supply nanodroplets, microbubbles, t-PA, and/or other blood thinner drug (e.g., pharmaceutical compound) outwardly of the first ultrasonic transducer arrangement 1 from the distal end 275 of the catheter 3. The catheter 3, first ultrasonic transducer arrangement 1, acoustic lens 5, and supply conduit 4 are described in detail above and therefore not described in further detail with respect to FIG. 3.

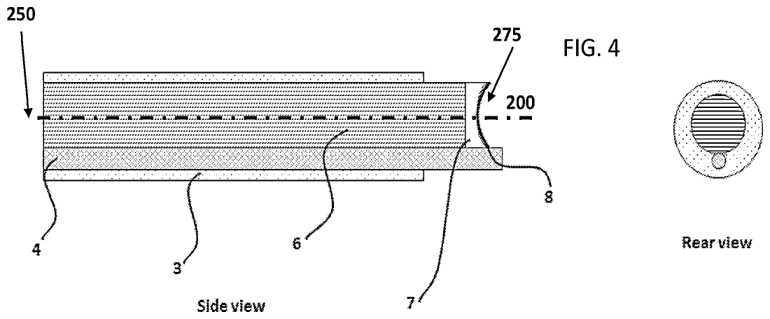

In another aspect, the present disclosure (see, e.g., FIG. 4) provides a front-firing, LGFU transducer element for intravascular thrombolysis. Such a device comprises a catheter 3 defining a longitudinal axis 200 and having opposed proximal and distal ends 250, 275. The device includes a laser ultrasonic transducer arrangement disposed about the distal end 275 and oriented with acoustic waves propagating parallel to the longitudinal axis 200. As described herein, the laser ultrasonic transducer arrangement includes a LGFU 7 lens and a laser ultrasound transduction layer 8. The device also includes a micro-optical fiber (or fiber bundle) 6 extending along the longitudinal axis 200 of the catheter 3 and into operable engagement with the LGFU lens 7. Additionally, a supply conduit 4 is arranged along the catheter 3 and is configured to supply nanodroplets, microbubbles, t-PA, and/or other blood thinner drug (e.g., pharmaceutical compound) outwardly of the laser ultrasonic transducer arrangement from the distal end 275 of the catheter 3. The catheter 3, micro-optical fiber 6, LGFU lens 7, laser ultrasound transduction layer 8, and supply conduit 4 are described in detail above and therefore not described in further detail with respect to FIG. 4.

More particularly, a catheter-mounted small aperture hybrid ultrasound transducer array is configured and arranged for ultrasound thrombolysis, in an approach with minimal use of a pharmacological agent. This device is capable of generating ultrasound or ultrasonic energy in axial and radial directions of the catheter when the transducer is close to a blood clot (see, e.g., FIG. 2 having first and second ultrasonic transducers 1 and 2, respectively). In some aspects, the catheter diameter is 2 mm and an external diameter of the transducer assembly is about 2 mm. In some instances, the catheter diameter is 2 mm or even larger. It should be understood that the dimensions for the catheter and/or transducer are provided only as examples and can have other values. Various combinations of forward-viewing piezo-transducer, side-viewing piezo-transducer, and forward-viewing LGFU-transducer are available. Six example configurations of IVUS transducers are: 1) a front-firing, piezoelectric stacked-type, flat or focused element (see, e.g., FIG. 3); 2) a front-firing, LGFU transducer element (see, e.g., FIG. 4); 3) combined front and side-firing piezoelectric transducer (see, e.g., FIG. 5); 4) a front firing piezoelectric ultrasound transmitter combined with a laser-generated focused ultrasound (LGFU) transducer (see, e.g., FIG. 6 excluding transducer 2); 5) front-firing LGFU combined with the side-firing piezoelectric elements (see, e.g., FIG. 6 excluding transducer 1); and 6) combined front-firing piezoelectric transducer, front firing LGFU element and side-firing piezo-elements (see, e.g., FIG. 6).

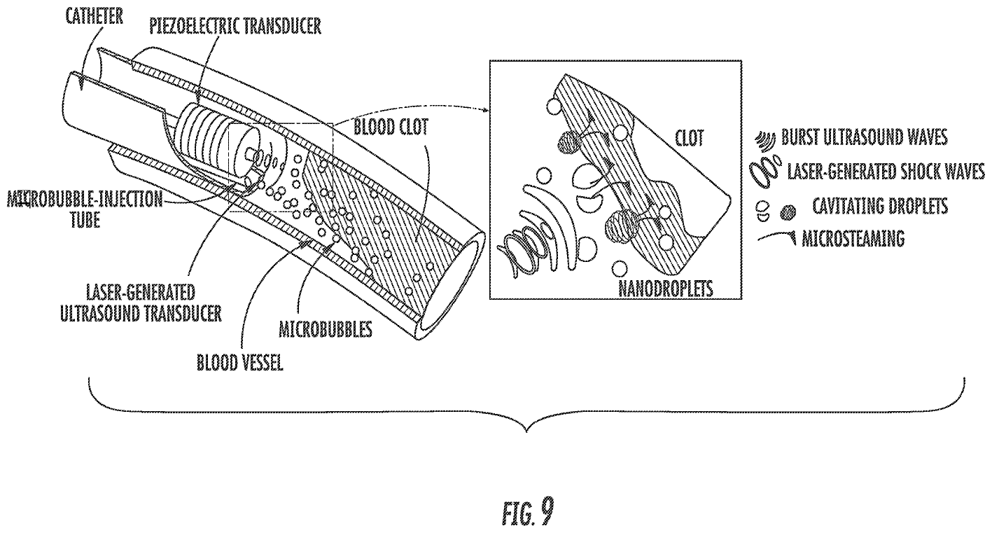

For both a piezoelectric and a hybrid laser-piezoelectric IVUS transducer, the front-firing element may have a multi-layer stacked structure (see, e.g., FIG. 8) for higher acoustic power, and smaller capacitance which leads to good electrical impedance matching with relatively low electrical impedance at the resonance of the transducer device. The total thickness of front firing transducer element is about 0.5 mm-<5 mm for the frequency range of <1 to 3 MHz, which is advantageous for efficient thrombolysis and microbubble excitation. The side-firing array transducer elements may have a single layer structure and operate in lateral mode. In one instance, due to the limited diameter of arteries (˜2 mm), the low-frequency thickness mode of side-firing elements (>1 mm thickness for <1 to 3 MHz) is difficult to achieve. Thus, as an example, ultrasound generation with a lateral mode (e.g., 1.9 MHz at 500 μm width) is practical for this application. Among many piezoelectric ceramics, crystals, and composites, PZT-2, PZT-5A, PZT-5H ceramics and single crystals including, for example, PMN-PT, PZN-PT and PIN-PMN-PT, generally show high ultrasound wave radiation along the thickness direction in the lateral mode. It should be understood that PZT-2, PZT-5A, and PZT-5H ceramic and/or PMN-PT, PZN-PT, or PIN-PMN-PT crystal are provided only as examples. This disclosure contemplates using other ceramics, crystals, and/or composites with the devices described herein.

The front-firing element of a hybrid IVUS transducer may be combined with a multi-layer stack piezoelectric transducer element and an LGFU lens. The LGFU lens may be comprised of a plano or a concave optical lens coated with carbon black and polydimethylsiloxane (PDMS), or carbon nano-fiber film and PDMS, or other light absorption materials and PDMS or other thermoelastic materials. In one example, a 532 nm laser light can be delivered through an optical fiber to the lens and the carbon-based material layer (e.g., carbon black, carbon nanotubes, carbon nano-fiber film, or carbon nano-particles) on the lens absorbs the light. The rapidly increased temperature due to the absorbed laser energy induces a rapid thermal expansion of the PDMS layer, and then a shock wave is generated outwardly of the front side of the lens. High amplitude shock waves can be achieved with high laser energy, and single-pulsed cavitation is also induced when the focal points of LGFU lens and the piezoelectric element are coincident. For both IVUS transducer arrangements, a micro-tube (e.g., supply conduit 4 in FIGS. 3-6) may be disposed inside the catheter to inject nanodroplet, microbubble, and t-PA agents (or other pharmaceutical compound) outwardly of the front firing transducer element to the treatment location.

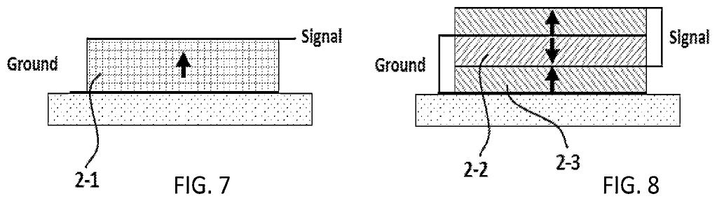

Characteristics of the catheter-mounted, small aperture, hybrid ultrasound transducers and arrays for intravascular thrombolysis can include one or more of the following: 1) a small aperture transducer fabricated small enough to fit within some space-limited application environments (i.e., within the catheter); 2) a transducer that can transmit ultrasound in a low frequency range (<1-3 MHz), which may be advantageous for thrombolysis efficiency and microbubble excitation by using multi-layer stacked thickness mode and lateral mode operation; 3) injection of nanodroplets/microbubbles (e.g., via supply conduit 4 in FIGS. 3-5) to the treatment position through a micro tube (e.g., supply conduit) inside and extending along the catheter, to relieve cavitation threshold PNP; 4) high pressure generation through both front and side firing transducer elements to induce the cavitation by using multi-layer stacked thickness mode and lateral mode operations; 5) a front-firing piezoelectric transducer element (e.g., component 1 in FIGS. 3, 5, and 6 and components 2-2 and 2-3 in FIG. 8), wherein the multi-layer stacked structure device is configured to achieve low-frequency operation and high pressure generation; 6) a front-firing LGFU lens (e.g., components 7 and 8 in FIGS. 4 and 6) having a carbon black, carbon nano-fiber film, or carbon nano-particles, combined with PDMS, to generate shock waves outwardly of the front side of the catheter, wherein the focal point shared with the piezoelectric element can efficiently induce the cavitation; 7) a micro-optical fiber (e.g., component 6 in FIGS. 4 and 6) implemented to deliver laser light to the LGFU lens; and/or 8) side-firing piezoelectric array elements (e.g., component 2 in FIGS. 5 and 6 and component 2-1 in FIG. 7), wherein PZT ceramics and/or piezoelectric single crystals are used as an element of a cylindrical array which is operable in a lateral resonance mode.

In one particular approach, ultrasound and laser ultrasound implemented in relation to thrombolysis, tissue ablation, and drug delivery, for example, have demonstrated cavitation enhancement and enhanced thrombolysis through a multi-frequency strategy. The multi-frequency strategy provides enhanced cavitation by using multi-frequency excitation, either through multiple piezoelectric transducers at frequencies <3 MHz or a laser-excited acousto-optic transducer. In this regard, a forward-looking multi-frequency catheter transducer for sonothrombolysis may be an advantageous configuration. The forward-looking transducer arrangement may, for example, facilitate ultrasound image guidance, reduce the amount of fluoroscopy required, limit the likelihood of catheter-clot contact, and direct acoustic energy forward towards the clot rather than directly towards the vessel wall. A combination of photo-acoustic and piezo transducers may provide both shock wave high frequency excitation and low frequency excitation, which may facilitate exciting of cavitation in microbubble agents. Certain data also suggests multi-frequency sonothrombolysis provides better clot dissolution performance over single frequency thrombolysis.

In addition, the catheter (e.g., component 3 in FIGS. 3-6) may also be configured to facilitate local administration, for example, of low-boiling point phase-change perfluorocarbon nanodroplets. Data suggests that sub-micron agents intercalate into clot matrices, and convert to cavitating microbubbles in response to acoustic energy, providing enhanced clot disruption over traditional microbubbles. Thus, aspects of the disclosure involve the development, optimization, and integration of several technologies for sonothrombolysis. Aspects of the DECLUT system can, for example, 1) improve lysis rate and significantly reduce treatment time, 2) reduce required pharmacologic lytic administration dose, thereby reducing off-target bioeffects, 3) reduce lysis fragment size, thereby reducing likelihood of downstream embolism, 4) reduce thermal and mechanical damage to off-target tissue, and/or 5) reduce fluoroscopy exposure to patient and caregiver.

Aspects of the present disclosure may thus implement low-frequency (<1 MHz-3 MHz) piezoelectric transducers for catheter-based sonothrombolysis by implementing small-aperture, low-frequency piezoelectric ultrasound transducers, with sufficient acoustic output for enhanced cavitation, into a 7-French or smaller catheter. In addition, nanodroplet formulation and size are optimized for clot-busting propensities, in conjunction with the ultrasonic energy. In addition, an optical fiber laser generated focused ultrasound (LGFU) transducer may be integrated into the catheter. When combined with the low frequency piezoelectric transducer, high-efficiency multi-frequency treatment may result. More particularly, combined excitation by low frequency continuous waves and LGFU shock waves, in addition to spatiotemporal delivery of t-PA and microbubbles/droplets, can provide quick and safe thrombolysis. For example, a miniaturized piezoelectric multifrequency ultrasound transducer (<1.5 mm in diameter) may be integrated in a catheter to generate cavitation-induced microstreaming, while an enhanced cavitation effect may be realized by using LGFU shock waves to cause inertial cavitation. Furthermore, forward-looking ultrasound waves provide ultrasound image guidance for clot detection without damaging intimal layers of vein walls. That is, a high-frequency (˜10 MHz) imaging piezoelectric transducer stacked in front of the low frequency therapy-transducer may provide image guidance, while minimal t-PA delivery combined with microbubbles/droplets reduce sizes of clot debris after the treatment to minimize the risk of recurrent and distal embolism. Finally, a 200 nanometer-diameter or smaller phase-change droplet agent formulation, converting to ˜1 micron microbubbles with reduced acoustic energy, will better penetrate clot matrices than standard microbubble formulations and cause optimally efficient thrombolysis. An exemplary specification for a DECLUT system as disclosed herein, is shown below in Table 1:

| | |
|---|---|
| External size | <7 French |
| Frequency | ~0.5 MHz-3 MHz for burst ultrasound |
| | (front firing and side firing) |
| | ~10 MHz for LGFU |
| | ~10 MHz single-pulse for A-mode imaging |
| | (forward looking) |
| Ultrasound output | MI of up to 1.9 for <1-3 MHz burst ultrasound |
| | MI of up to 1.9 for ~10 MHz LGFU |
| Focal length | <1.5 mm |
| −6 dB focal spot size | <3 mm in axial direction |
| | <1.5 mm in lateral direction |
| t-PA dose | <100 μg |
| Lytic rate | >3% mass loss/min |

Aspects of a DECLUT system, as disclosed herein, may thus advantageously realize, for example, 90% dissolution in 30 minutes (3% mass loss/min) with the use of t-PA of <100 g, as compared to existing sonothrombolysis techniques (e.g. EKOS) which needs >15 hours for complete lysis (approximately 0.11% mass loss/min) with the use of t-PA of 10-20 mg. Accordingly, faster (i.e., >10 times) clot dissolution is achieved compared to current sonothrombolysis approaches (e.g. EKOS) through the combined mechanism of ultrasound-mediated fibrinolysis and micro-fragmentation arising from cavitation-induced microstreaming at a reduced cavitation threshold, which is attributed to the MCA/droplet and dual-ultrasound excitation. Moreover, safer clot-dissolution may be realized over current catheter-based thrombolysis techniques (e.g. Angiojet, Trellis, and EKOS) due to, for instance, the minimal use and precise delivery of lytic agent, and reduced physical contact to the target clot and the acoustic exposure of the surrounding vessel wall. In instances where implemented, forward-looking ultrasound image guidance will to help reduce fluoroscopy exposure to patient and caregiver.

In some aspects, the ultrasonic transducer(s) is/are used to excite the injected microbubble contrast agents (MCA) or nanodroplets to cause enhanced cavitation-induced microstreaming. These low-frequency (<1 MHz-3 MHz) miniaturized (<1.5 mm) piezoelectric transducers or arrays thereof may be configured as multi-layer structures and/or to be operable in a lateral mode. Moreover, the tightly focused high-pressure shock wave excitation provided by the LGFU transducer is utilized for intravascular thrombolysis. For the higher lytic rate, these two different forward looking transducers may share the same focal spot, enhancing cavitation effects due to the reduced cavitation pressure threshold by dual-sonication. Although sufficient lytic rate can be expected without t-PA injection for this DECLUT system, reducing the risk of bleeding complications, minimal t-PA dose can eliminate the risk of potential recurrent or distal embolism which could occur due to clot debris, as with current systems. The integrated device will be located approximately >1 mm away from the target clot, and hence there is no direct contact between the device and the clot, which may enhance the safety of the device/procedure and still allow precise spatiotemporal delivery of t-PA and microbubbles/droplets.

For low-frequency ultrasound excitation with sufficient conditions for cavitation, the piezoelectric transducer(s) can be configured to account for spatial limitations (e.g., an aperture of <1.5×1.5 mm²). Thus, a multi-layer stacked longitudinal-mode resonator (electrical field and wave propagation are both along the catheter axial direction) and/or a lateral-mode resonator (electrical field is perpendicular to the catheter axial direction, while the acoustic wave propagates along the axial direction) may be implemented. The total thickness of a longitudinal mode transducer may be greater than about 1.5 mm such that the transducer has a resonance frequency lower than 1 MHz. However, the achievable acoustic output of a monolithic piezoelectric bulk element is limited, due to low capacitance, low strain and the driving voltage limitation. The multi-layer stacked configuration has electrically-parallel and mechanically-serial connection of stacked elements, which provides a more efficient ultrasonic transducer transmitter with lower electrical impedance, higher strain and the capability of multi-frequency modes. For the lateral-mode transducer, the lateral-resonance frequency is dependent on the lateral dimension (perpendicular to the electrical field), and is independent of the thickness (parallel to the electrical field). Thus, the thickness of the lateral mode transducer can be configured with lower electrical impedance. Both the multi-layer stacked and lateral mode transducers exhibit a low operating frequency (<1 MHz) and multi-frequency ultrasound within a <7-french catheter as well as acceptable electrical impedance (<500 ohm) at the resonance frequency for forward looking and side looking high intensity ultrasound-induced cavitation. Moreover, the high frequency (10 MHz) forward looking ultrasound image can be used to guide the positioning of the catheter, while reducing the fluoroscopy exposure for the practitioner.

The high-pressure output at the tight focal spot of the LGFU arrangement may also be utilized for intravascular thrombolysis. A miniaturized carbon nanoparticle (CNP)/PDMS LGFU transducer implements an optical fiber for exciting microbubbles with high-pressure (>MPa) shock waves, which is difficult to achieve with miniaturized piezoelectric ultrasound transducers. The pressure output of the LGFU arrangement at the focal spot is sufficient to drive substantial microbubble cavitation and microstreaming in as focused manner in proximity to the target clot, while minimizing the potential risk of vessel injury due to the tight focal spot size (<2 mm in axial direction and <1 mm in lateral direction) of a fiber LGFU transducer/arrangement.

Enhanced cavitation by dual-acoustic excitation may be useful for therapeutic ultrasound applications as well as thrombolysis. Combining the high frequency shock waves generated by the LGFU transducer/arrangement and low-frequency burst waves generated from the piezoelectric ultrasound transducers are applied for thrombolysis with higher efficiency, wherein the dual-acoustic excitation can result in a higher lytic rate than conventional ultrasound-mediated fibrinolysis, such as EKOS (i.e., treatment time >15 hours in average). Low-boiling point phase change contrast agents may comprise, for example, liquid perfluorobutane nanodroplets which vaporize into microbubbles upon interaction with acoustic energy. Such low boiling point perfluorocarbon can be vaporized at even low acoustic pressures (less than a MI of 1.9), whereas traditional perfluoropentane or perfluorohexane nanodroplets require substantially higher energy levels to phase convert, due to Laplace pressure and homogeneous nucleation. These liquid perfluorobutane nanodroplets are very stable in liquid precursor form and are thus relatively robust and able to withstand high hydrostatic pressure and shear that occurs when pumping bubbles rapidly down a long small-bore of a catheter to the treatment site. Furthermore, these droplets can be readily configured in the <100-300 nanometer size range, for improved clot penetration compared to <1-3 micron bubbles while achieving smaller debris fragment size. Upon activation by ultrasonic energy, the resulting microbubbles behave similarly or identically to traditional microbubbles, but may result in improved clot lysis due to clot intercalation.

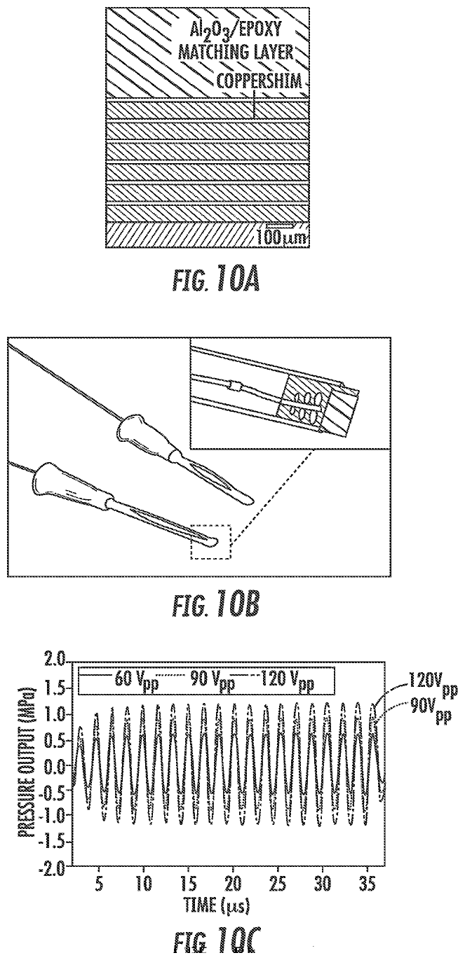

In some aspects, a small-aperture, low-frequency piezoelectric ultrasound transducer may be formed and configured with sufficient acoustic output (MI˜0.3-1.9) for enhanced cavitation in a 7F catheter. A multi-layer stacked design may improve power transfer efficiency of the transducer in transmit mode. Multi-layer transducers are also able to increase element capacitance by a factor of N² since they are stacked mechanically in series and electrically in parallel, where N is the total number of layers, which has significant effects on the transducer transmitting sensitivity. That is, the power output Pₒᵤₜ=Vₒᵤₜ²/Rₘ is maximized when the mechanical resistance Rₘ is minimized, given the equation of Rₘ,

$${R}_{m} = \frac{π}{4 {k}_{eff}^{2} ω {C}_{0}} {Z}_{a}$$

where k_{eff} is the electromechanical coupling of the piezoelectric, C₀ is the static element capacitance, and Zₐ is the ratio of front acoustic loads to that of the piezoelectric element. Thus, in a multilayer transducer, the Rₘ is decreased by a factor of N², resulting in an equal increase in power output. Therefore, multi-layering can significantly reduce the transmit voltage of the transducer for the same output pressure. A comparison between a single layer and a 5-layer PZT 2D array found that a ˜5.6 dB transmitting efficiency gain could be obtained with the 5-layer design. In one instance, a miniaturized, low-frequency, high-power transducer was implemented for MCA-involved sonothrombolysis, the transducer array comprising PZT-5A 6-layer transducers with an aperture of 1.2×1.2 mm² and the total thickness of 1.7 mm, and exhibited a longitudinal-extensional-mode resonance frequency of 550 kHz (see, e.g., FIGS. 10A-10C). The achieved peak-to-peak pressure output was about 2.2 MPa at the driving voltage of 120 Vpp (FIG. 10C). The PNP was about 1 MPa and the corresponding MI was 1.4, which is sufficient for cavitation-induced microstreaming.

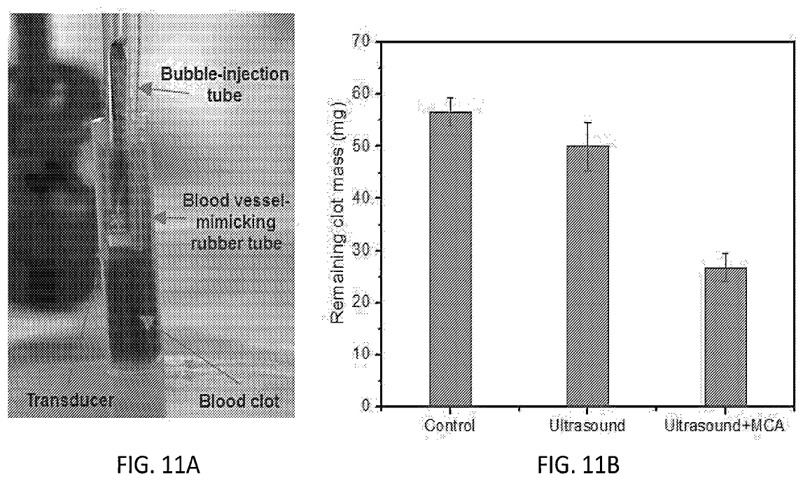

The exemplary transducer was then implemented in in vitro thrombolysis tests (FIG. 11A). A microbubble-injection tube was integrated with the transducer, and the transducer-tipped needle was positioned 1 mm away from the target blood clot stored in the saline water-filled vessel-mimicking tube (inner diameter of 3 mm). The blood clot was exposed to the low-frequency (550 kHz) ultrasound with a duty cycle of 7% (300 cycle burst with 5 ms of pulse duration). The treatment time was 30 min, and the lytic rate of treatment cases with and without MCA were compared. For the bubble injection case, microbubbles were injected at a concentration of 1×10⁸/mL and at a flow rate of 0.1 ml/min. As shown in FIG. 11B, ultrasound treatment with MCA shows the clot mass reduction of 50%, whereas ultrasound excitation alone without MCA showed less than 10% clot mass loss. Thus, these results suggest that the low frequency multi-layer transducer with a small aperture (1.2×1.2 mm²) can be used to generate sufficient acoustic output for effective MCA-mediated thrombolysis. The achieved lytic rate with MCA was 1.67%/min, though a higher lytic rate may be achieved with the use of t-PA, because other studies indicate that MCA-involved sonothrombolysis with the use of 0.32 g/mL t-PA improves the lytic rate ˜5× more than the same treatment without t-PA.

Another advantage of a multi-layer stacked design is that multi-frequency operation can be realized. More particularly, in one instance, a single-aperture, dual-layer HIFU transducer (diameter of 25 mm) was implemented to operate at 1.5 MHz and 3 MHz, simultaneously. The transducer has half-wavelength and quarter-wavelength resonance modes at frequencies of 1.5 MHz and 3.1 MHz, respectively. Efficacy of dual-frequency excitation showed a 5% higher cavitation-induced temperature increment for tissue ablation, wherein the mechanism of the improvement is the reduced threshold pressure for cavitation with dual-frequency excitation. In another instance, dual-frequency excitation for TDEU thrombolysis was implemented to reduce the required acoustic power for sonothrombolysis. The 1.5 MHz HIFU transducer was used, and the multi-frequency excitation case (e.g. 1.4 MHz+1.5 MHz) was compared with the single-frequency excitation (1.5 MHz) case. The dual-frequency ultrasound was able to accelerate the lytic rate by a factor of 2-4 compared to the single frequency case. No significant differences were found between dual-frequencies with different frequency differences (0.025, 0.05, and 0.1 MHz), or between dual-frequency and triple-frequency.

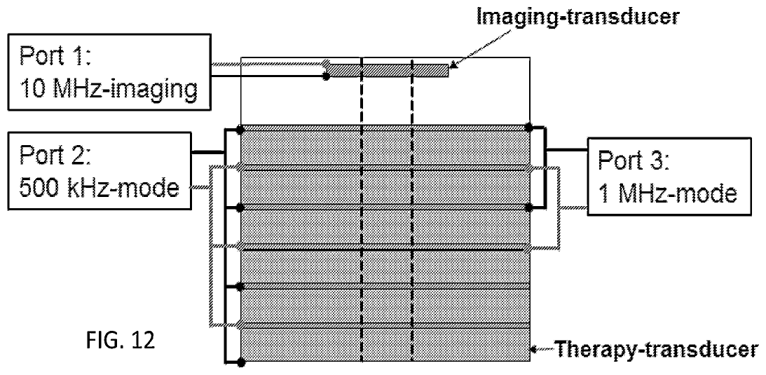

In dual-frequency therapy transducer design, half-wavelength resonance frequency is determined by the total thickness of the stacked-layers. Once the total-thickness frequency is selected, the quarter-wavelength resonance frequency is determined as twice of the half-wavelength case (FIG. 12). Although the frequency components are determined by the 1-dimensional analysis for the wave propagation along the thickness direction, the proper number of layers, the achievable pressure output, the corresponding MI, and the beam profile at each frequency with a given electrical input, must all be analyzed and optimized, for example, by finite element analysis (FEA), and the optimal dimension determined based on the FEA results. For example, ANSYS FEA software (ANSYS Mechanical APDL, ANSYS Academic Research, Release 15.0.7, ANSYS, Inc., Canonsburg, Pennsylvania USA) can accurately simulate acoustic performance of the stacked-type multilayer transducers, and can be used to optimize design factors such as total thickness, number of layers, and aperture size for the aimed beam diameter (<1 mm) and MI (>1.0) at the target distance (>1 mm). Generally, lower-frequency ultrasound excitation realizes a higher lytic rate. However, the lower frequency ultrasound beam has a larger beam width, though focal spot size and beam profile are important design factors for forward-looking intravascular therapeutic-ultrasound transducers, since the redundant ultrasound beam may cause ultrasound-associated vascular injury. As such, the beam width of burst-waves in a DECLUT catheter can be optimized by using a customized concave lens. Generally, a −6 dB beam width can be approximately estimated by the equation,

$$B {D}_{- 6 D b} \approx 1.41 ( R / D ) ( c / f )$$

where R, D, c, and f denote a radius of the curvature of a concave lens, the diameter of the lens, the wave velocity of the medium, and the operating frequency, respectively. With the aperture of 1.2×1.2 mm² at the operating frequency of 500 kHz and 1 MHz for the 1 mm focal distance, the −6 dB beam diameter for each frequency can be approximately calculated as 3 mm and 1 mm, respectively. Based on the target size, proper lens material and radius of curvature can be optimized, and the corresponding focal gain, −6 dB beam width, and focal spot size can be determined. The specifications of a dual-frequency, multi-layer transducer is shown, for example, in Table 2:

| | |
|---|---|
| Aperture | 1.2 × 1.2 mm² |
| # of layer | ~6 layers |
| Impedance at resonance | <100 Ω at both resonance frequencies |
| Frequency | A-mode imaging: >10 MHz |
| | Sonothrombolysis: <1-3 MHz |
| Ultrasound output | MI of-up to 1.9 |
| | (FDA diagnostic ultrasound limit is 1.9) |
| Focal length | >1 mm |
| −6 dB focal spot size | ~2 mm in axial direction |
| | <vessel diameter |

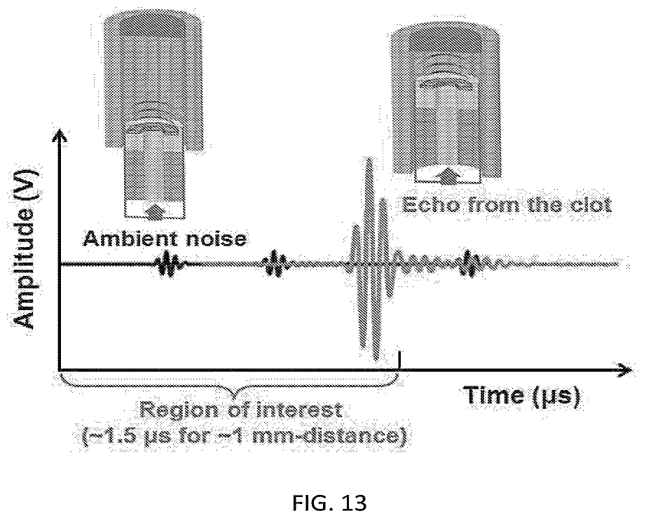

For the high-frequency (>10 MHz) imaging transducer, pulse-echo response can be estimated by KLM modeling, and it is expected that A-mode imaging is available by way of the imaging transducer disposed in front of the low-frequency therapy transducer (FIG. 13). It has been shown that a high-frequency (>12 MHz) transducer in a stacked-type multi-frequency transducer did not affect the transmitting performance of the low-frequency (2 MHz) transducer.

For a multi-layer stacked configuration transducer, piezo plates (e.g. PZT-2 having an area of 5×5 mm² and thickness of 250-350 μm) can be stacked with a 20 μm-thick copper shim between adjacent piezo plates. The quarter-wavelength matching layer can be made of alumina powder/epoxy bond mixture with an acoustic impedance of ˜7-8 MRayl is attached at the front side. After bonding of the layers, the assembly is diced to obtain an aperture of 1.2×1.2 mm². The transducer(s) are wire-connected and mounted in a 7F catheter as a forward-looking transducer arrangement. The resulting multi-layer transducers exhibit multi-frequency modes, reasonably high sensitivity and bandwidth at high frequency for imaging guidance, and sufficient MI for enhanced cavitation. The multi-layered transducer configuration with the small aperture for mounting in a 7F catheter generally requires a small bonding area to maintain sufficient bonding condition.

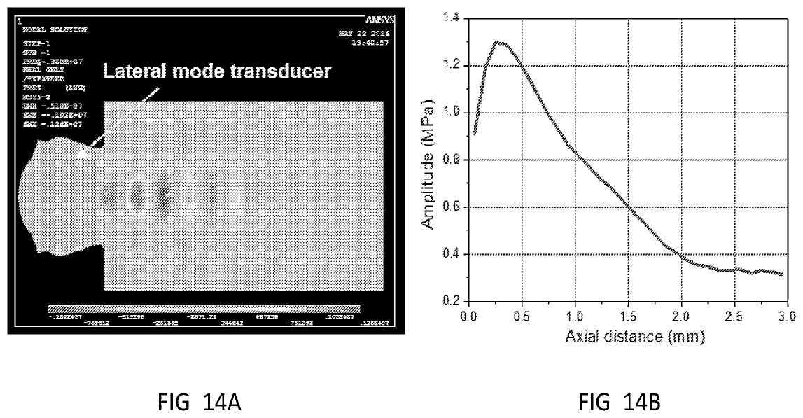

The low-frequency transducer for a DECLUT system may also be configured as a lateral-mode transducer where the resonance frequency is determined by the lateral dimension and is the operating frequency. Once the lateral dimension is determined (i.e., 1.2 mm), the usual piezoelectric lateral mode frequency is in the range of 1-2 MHz, which is independent of the thickness as long as the lateral dimension is at least 3 times larger than the thickness. In one example, a relatively small size (1.2×1.2×0.3 mm³) PZT-5H lateral mode transducer can generate about 1 MPa PNP output with 100 Vpp sinusoidal excitation at 1.5 MHz lateral mode frequency (see, e.g., FIGS. 14A and 14B).

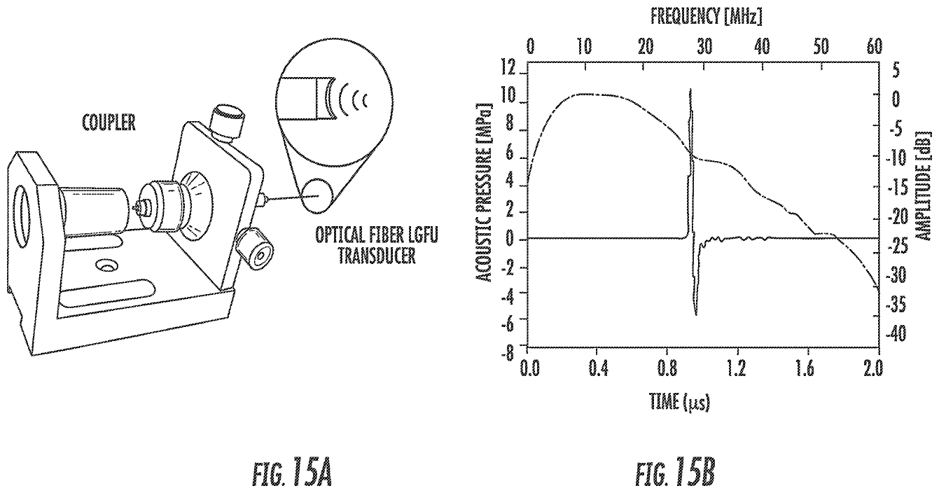

Optical fiber LGFU transducers are fabricated from CNP/PDMS composite film and such miniaturized LGFU transducers are integrated into a 7 French catheter for thrombolysis. A laser ultrasound transducer comprised of a CNP/PDMS composite film can be prepared using a candle soot process. In comparison with other carbon-based composite films (e.g., carbon-black, carbon-nanotube, carbon-nanofiber with PDMS layer), the CNP/PDMS film exhibits a higher light-to-acoustic energy conversion ratio due to a higher light absorption coefficient and a faster heat transfer characteristic due to a low interfacial thermal resistance. Moreover, the CNP/PDMS film can be formed through a relatively easy and cost-efficient candle soot fabrication process. The miniaturized LGFU transducers for catheter thrombolysis (CTTU) can comprise an optical fiber LGFU transducer prepared using a CNP/PDMS film (FIG. 15A). In one example, an optical fiber (0.6 mm in diameter) CNP/PDMS LGFU with a lens (1 mm in diameter) can generate a high-pressure shock wave (peak to peak pressure of 16 MPa with 11 MHz center frequency) at 1 mm away from the transducer (FIG. 15B). The implemented laser input was only 1.5 mJ of a 532 nm pulsed laser, and thus higher pressure output can correspond to higher laser input.

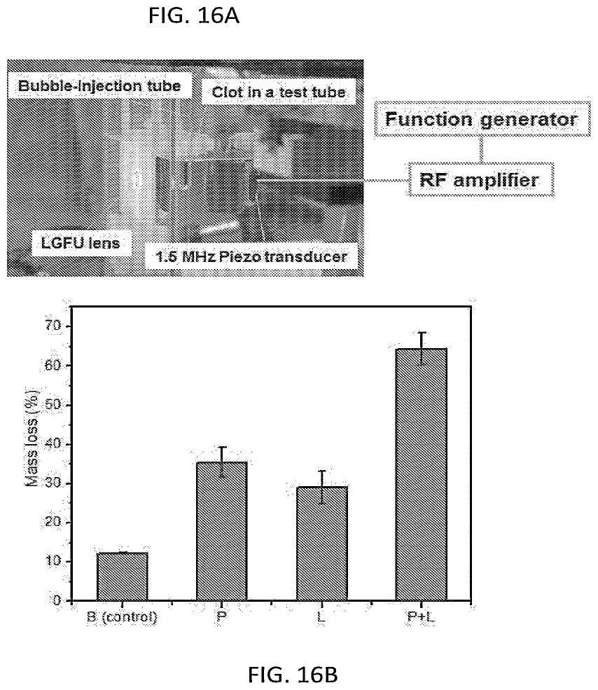

An initial in vitro test was used to evaluate the lytic efficiency of the dual-excitation of LGFU and low-frequency burst ultrasound. In the initial test, a LGFU transducer (diameter of 12 mm and radius-of-curvature of 12.4 mm) and a piezoelectric transducer (1.5 MHz, diameter of 30 mm and focal length of 30 mm) were used to evaluate the feasibility of dual excitation for thrombolysis regardless of size and catheter design. The LGFU transducer was comprised of carbon-black and PDMS, and the peak frequency was 11 MHz. The experimental arrangement is as shown in FIG. 16A. Three different treatment cases were compared: the treatments with 1) piezo transducer only, 2) LGFU only, and 3) dual-excitation of piezo transducer and LGFU. Each treatment time was 15 minutes. The test result is shown in FIG. 16B. The case of dual-excitation exhibited higher mass loss than the other two cases: 85% higher than piezo transducer treatment only and 100% higher than LGFU treatment only. These results demonstrate that the dual-excitation of LGFU and low-frequency burst waves is beneficial to MCA-involved sonothromobolysis due to the enhanced cavitation effect, and therefore the overall lytic rate can be significantly improved with the use of small dose of t-PA (<0.3 g/mL).

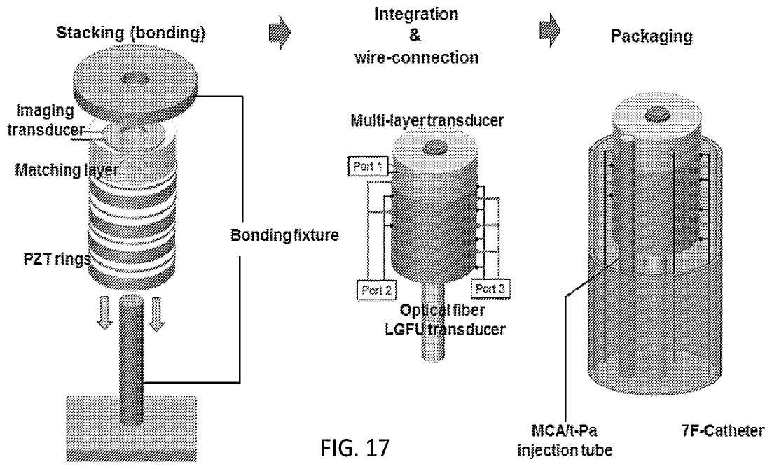

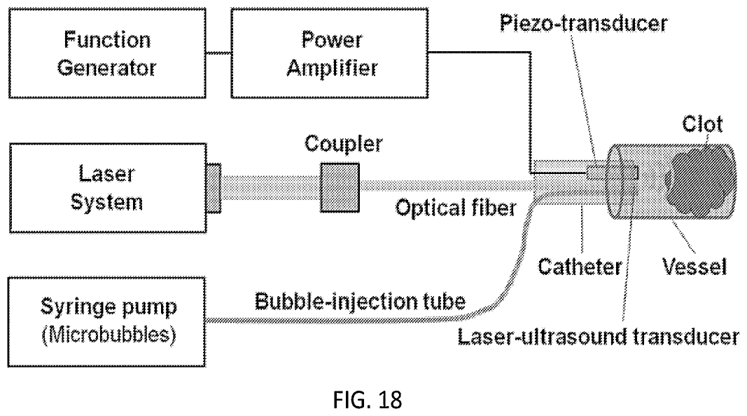

A PDMS concave lens can be fabricated by using the capillary effect of uncured PDMS at the top of a plastic tube having an inner diameter of 0.8 mm. After curing the PDMS lens, a CNP layer can be deposited on the concave surface by a candle-soot process. A PDMS thermal expansion layer can be coated on the CNP layer by dip-coating. The fabricated LGFU lens has a diameter of 0.5 mm and a radius-of-curvature of about 1 mm. A 0.3 mm-diameter optical fiber is attached to the LGFU lens by using optical glue. The integration of the LGFU transducer with the multi-layer transducer can be processed as shown in FIG. 17. A microtube (ID: 0.3 mm, OD: 1 mm) for injecting the microbubble and t-PA can be attached at the side of the integrated transducer, and the integrated assembly mounted on the tip of a 7F catheter. The optical fiber LGFU transducer is mounted on a fiber-coupler (FIG. 18), because the initial beam diameter of a 532 nm Nd:YAG pulsed laser (Minilite I, Continuum Inc., Santa Clara, CA) is about 10 mm. FIG. 18 shows the integrated DECLUT system.

Aspects of the present disclosure thus combine and cooperate to provide a device having a low-frequency (<1 MHz), miniaturized (<1.5 mm in diameter), high acoustic output (MI of 0.3-1.9) multi-frequency intravascular piezoelectric ultrasound transducer for forward looking image guided intravascular thrombolysis. Optical fiber CNP/PDMS LGFU transducers generate high-pressure (<5 MPa-20 MPa) shock wave to enhance cavitation-induced microstreaming near the clot. Combined t-PA and MCA/nanodroplets reduce required acoustic energy and improve lytic rate. Dual-excitation of the blood clot by LGFU shock waves and burst waves by the piezoelectric ultrasound transducer leads to enhanced cavitation at a tight focal spot (a fraction of a vessel diameter) while reducing potential risk of injury to the vessel wall. Low-boiling point phase change agents further serve as a microbubble thrombolysis source, but provide improved stability for inter-catheter delivery and improved clot penetration and subsequent lysis.

1. A catheter-implemented transducer device for intravascular sonication, comprising:
a catheter defining a longitudinal axis and having opposed proximal and distal ends;
an ultrasonic transducer arrangement disposed about the distal end of the catheter, the ultrasonic transducer arrangement comprising a plurality of piezoelectric transducers, wherein the plurality of piezoelectric transducers arranged electrically in parallel and mechanically in series; and
a supply conduit arranged along the catheter, the supply conduit configured to supply contrast agents from the distal end of the catheter,
wherein the ultrasonic transducer arrangement is configured to generate pressure to cavitate the contrast agents.

2. The device of claim 1, wherein the ultrasonic transducer arrangement is configured, in a lateral mode, to emit a forward viewing low-frequency ultrasonic energy that generates pressure.

3. The device of claim 2, wherein the ultrasonic transducer arrangement is configured, in a longitudinal mode, to emit a forward viewing low-frequency ultrasonic energy that generates pressure.

4. The device of claim 1, wherein the ultrasonic transducer arrangement is configured to emit ultrasonic energy with a frequency of less than 1 MHz.

5. The device of claim 1, wherein the supply conduit is further configured to deliver a lytic agent.

6. The device of claim 1, wherein the ultrasonic transducer arrangement is configured to have an acoustic output with a mechanical index of greater than 0.3.

7. The device of claim 1, further comprising a multi-layer stacked longitudinal-mode resonator.

8. The device of claim 1, wherein the ultrasonic transducer arrangement has a thickness greater than 0.5 mm.

9. The device of claim 1, wherein the ultrasonic transducer arrangement has a resonance frequency less than 1 MHz.

10. The device of claim 1, wherein the ultrasonic transducer arrangement is configured to operate at multiple frequencies simultaneously.

11. The device of claim 1, wherein the contrast agents comprise microbubble contrast agents.

12. The device of claim 1, further comprising a plurality of ultrasonic transducer arrangements on the distal end of the catheter.

13. A catheter-implemented transducer device for intravascular sonication, comprising:
a catheter defining a longitudinal axis and having a proximal end and a distal end;
an optical fiber configured to direct laser light along the longitudinal axis of the catheter;
a laser-generated focused ultrasound lens on the distal end of the catheter, and
a supply conduit arranged along the catheter, the supply conduit configured to supply contrast agents from the distal end of the catheter,
wherein the laser-generated focused ultrasound lens is configured to absorb the laser light from the optical fiber, thermally expand, and generate a shock wave of pressure to cavitate contrast agents.

14. The device of claim 13, further comprising an ultrasonic transducer arrangement disposed about the distal end of the catheter.

15. The device of claim 14, wherein the ultrasonic transducer arrangement is configured to emit ultrasonic energy with a frequency of less than 3 MHz.

16. The device of claim 13, wherein the supply conduit is further configured to deliver a lytic agent.

17. The device of claim 13, wherein the laser-generated focused ultrasound lens is a plano optical lens.

18. The device of claim 13, wherein the laser-generated focused ultrasound lens is a concave optical lens.

19. The device of claim 13, wherein the laser-generated focused ultrasound lens comprises a laser ultrasound transduction layer configured to absorb laser light, the laser ultrasound transduction layer comprising light absorption layer.

20. The device of claim 19, wherein the laser ultrasound transduction layer comprises a thermal expansion layer.

21. The device of claim 19, wherein the laser ultrasound transduction layer comprises a composite film of CNP and PDMS.

22. The device of claim 14, wherein the ultrasonic transducer arrangement is configured to cause cavitation and microstreaming in a focal spot of less than 2 mm in an axial direction and less than 1 mm in a lateral direction.

23. The device of claim 13, wherein the fret contrast agents comprise microbubble contrast agents.

24. The device of claim 13, further comprising a plurality of ultrasonic assemblies on the distal end of the catheter.

25-57. (canceled)
